(19) United States
(12) Patent Application Publication
(10) Pub. No.: US 2010/0029012 A1
(43) Pub. Date: Feb. 4, 2010

(54) Method for identifying modulators of the NRF2-KEAP1-AREP pathway

(76) Inventors: Jonathan T. Kern, Souderton, PA (US); John W. Hess, Lansdale, PA (US); Geeta Kandpal, Blue Bell, PA (US); Ian J. Reynolds, Collegeville, PA (US)

Correspondence Address: MERCK AND CO., INC, P O BOX 2000, RAHWAY, NJ, 07065-0907, US

(21) Appl. No.: 12/311,644

(22) PCT Filed: Nov. 2, 2007
(86) PCT No.: PCT/US07/23171

§ 371 Date: Apr. 7, 2009

Related U.S. Application Data
(60) Provisional application No. 60/857,078, filed on Nov. 6, 2006.

(51) Int. Cl. G01N 33/53

(52) U.S. Cl. 436/501

(57) ABSTRACT

A method for identifying modulators of the Keap1-NrG-ARE pathway is described. In particular, an assay is described that identifies molecules that inhibit the binding of a labeled Nrf2 peptide with the kelch domain of the Keap1 protein. Molecules that inhibit the binding are activators of the Keap 1-Nrf2-ARE pathway. Activation of the Keap 1-Nrf2-ARE pathway may result in an increased accumulation of Nrf2 and the subsequent induction of protective enzymes, for example, the phase 2 detoxification enzymes. Activators of the Keap1-NrG-ARE pathway are useful for combating oxidative stress-related disorders, such as those associated with cancer, emphysema, Huntington's disease, light-induced retinal damage, and stroke.

### BACKGROUND OF THE INVENTION

(1) Field of the Invention

The present invention relates to a method for identifying modulators of the Keap1-Nrf2-ARE pathway. In particular, the present invention relates to an assay for identifying molecules that inhibit binding of a labeled Nrf2 peptide with the kelch domain of the Keap1 protein. Molecules that inhibit binding of the labeled peptide to the Keap1 protein can be activators of the Keap1-Nrf2-ARE pathway. Activation of the Keap1-Nrf2-ARE pathway results in an increased accumulation of Nrf2 and the subsequent induction of protective enzymes, for example, the phase 2 detoxification enzymes. Activators of the Keap1-Nrf2-ARE pathway are useful for combating oxidative stress-related disorders, such as those associated with cancer, emphysema, Huntington's disease, light-induced retinal damage, and stroke.

(2) Description of Related Art

Oxidative stress is a well-studied, but poorly controlled, component of cellular toxicity in which highly reactive molecules damage DNA, proteins, and lipids. An imbalance between prooxidant species (including superoxide anion, peroxynitrite, and the hydroxyl radical) and the body's antioxidant defenses can lead to disruption of cellular function and may contribute to disease initiation and progression. Not all free radical production is deleterious to the body, however. Myeloperoxidase catalyzes the production of hypochlorous acid from hydrogen peroxide and chloride anion during the neutrophil's respiratory burst (Klebanoff, J. Leukoc. Biol. 2005, 77:598-625). This rapid, localized release of reactive oxygen species is critical to humans in the protection from bacteria; myeloperoxidase deficiency may result in a compromised immune response or higher incidence of cancer (Lanza, J. Mol. Med. 1998, 76:676-681). Thus, free radical production and quenching must be closely regulated so as to be permissive of desirable reactive oxygen species functions without allowing excessive free radical accumulation. It has been proposed that the cell accomplishes this feat through the compartmentalization of free radicals into “microdomains” (Terada, J. Cell Biol. 2006, 174:615-623), which would suggest that the cell has evolved a cytoplasmic redox sensing mechanism to identify and trigger an antioxidant response to quench the excessive accumulation or release of prooxidant species from their restricted locations.

Exogenous antioxidant therapies have been proposed to restore the redox balance of the cell. Unfortunately, clinical efforts utilizing exogenous antioxidant therapies have, to date, generated only modest or ambiguous results. These results would indicate the complexity of exogenous antioxidants as therapeutics, and may indicate the need to utilize a more refined method of combating oxidative stress. Although a complete listing of clinical trials involving antioxidant therapies is beyond the scope of this description of the related art, an examination of clinical trials involving alpha-tocopherol demonstrates the disappointing results achieved with exogenous antioxidant therapy. In reviews of completed clinical trials evaluating alpha-tocopherol therapy in cardiovascular disease, cancer, Parkinson's, Alzheimer's, tardive dyskinesia, and cataract, the available data did not justify a recommendation for use of the antioxidants for disease prevention, with the potential exception of alpha-tocopherol supplementation for patients with recently diagnosed tardive dyskinesia (trials reviewed in Pham et al., Ann. Pharmacother. 2005, 39:1870-1878; Pham et al., Ann. Pharmacother. 2005, 39:2065-2072). In fact, the use of some exogenous antioxidant therapies has been shown to worsen outcome in certain patient populations. In a clinical trial involving 20 mg per day beta-carotene supplementation, the researchers observed a higher incidence of lung cancer among those receiving beta-carotene compared to those who did not (The Alpha-Tocopherol, Beta Carotene Cancer Prevention Study Group. N. Engl. J. Med. 1994, 330:1029-1035). It has been proposed that clinical efforts involving high doses of antioxidants may interfere with cell functions involving redox signaling. A recent report of a clinical trial involving long-term dosing of a cocktail of antioxidants given at low, nutritionally-available doses demonstrates a decreased risk of cancer in males, but not in females (Hercberg et al., Arch. Intern. Med 2004, 164:2335-2342). It has been proposed that this gender bias is due to the lower basal levels of endogenous antioxidants in the male cohort when compared to the females (Galan et al., Br. J. Nutr. 2005, 94:125-132). These various clinical efforts suggest that the “tone”, or amount of antioxidant (whether exogenous or endogenous), along with an understanding of the pharmacokinetic parameters of the antioxidant used will be critical to the success of any therapeutic.

The antioxidant response element (ARE) is a cis-acting regulatory element found in the 5′-flanking region of genes encoding a number of cytoprotective enzymes and regulates the expression of these proteins in response to oxidative stress (Rushmore et al., J. Biol. Chem. 1991, 266:11632-11639). The coordinate upregulation of these genes results from the phosphorylation and translocation of the transcription factor Nuclear Factor Erythroid 2-like 2 (Nrf2) to the nucleus in response to stress on the cell. Once in the nucleus, Nrf2 forms a complex with small musculoaponeurotic fibrosarcoma oncogene (Maf) proteins and other components of the transcriptional machinery to induce expression of ARE-containing promoters (Itoh et al., Mol Cell Biol 1995, 15:4184-4193; Itoh et al., Biochem Biophys Res Commun 1997, 236:313-322; Moi et al., Proc. Natl. Acad. Sci. USA 1994, 91:9926-9930). Under basal conditions, Nrf2 is bound to Keap1, a cysteine rich E3 ubiquitin ligase substrate adaptor protein that is part of the ubiquitin-proteosome degradation pathway.

The Keap1 protein is comprised of several distinct domains; an N-terminal region of 66 amino acids, a BTB domain from amino acid residue 67 to 178, a 137 amino acid BACK domain (Stogios and Prive, Trends Biochem. Sci. 2004, 29:634-637) also known as the linker, central linker domain, or intervening region (IVR), a kelch domain comprised of amino acid residues 322 to 608, and a C-terminal region of 15 amino acids. Systematic deletion of each of these domains reveals that the kelch domain is required to bind and sequester Nrf2 and that the BTB and BACK domains are required for the modulation of Nrf2 levels by chemical agents (Zhang and Hannink, Mol. Cell. Biol. 2003, 23:8137-8151). In addition, the BTB domain of Keap1 is critical for dimerization of Keap1 (Zipper and Mulcah, J. Biol. Chem. 2002, 277:36544-36552). Several groups have provided convincing evidence demonstrating that Keap1 acts as a substrate-specific adaptor in an E3 ubiquitin ligase complex that ubiquitinates and targets Nrf2 for degradation by the proteosome (Furukawa and Xiong, Mol. Cell. Biol. 2005, 25:162-171; Kobayashi et al., Mol. Cell. Biol. 2004, 24:7130-7139; Zhang et al., Mol. Cell. Biol. 2004, 24:10941-10953). The ubiquitin-proteosome system comprises one of nature's most oft-repeated means of regulating protein levels within the cell. Three component enzymes, an E1 ubiquitin-activating enzyme, an E2 ubiquitin-conjugating enzyme, and an E3 substrate adaptor protein complex work in conjunction to covalently attach ubiquitin to a substrate. Subsequent to the first ubiquitin addition, a polyubiquitin chain may be synthesized. These ubiquitinated substrates are then targeted for degradation by the 26S proteosome. Cul3 (Cullin 3) is a core scaffolding protein in the E3 ligase complex and has direct protein:protein interactions with both Keap1 and Rbx1 (Ring box 1). Cul3 and Rbx1 form the catalytic component of the enzyme complex and interact with an E2 ubiquitin ligase to transfer ubiquitin to the substrate.

Under oxidative load, Nrf2's degradation by Keap1 is disrupted, resulting in the nuclear accumulation of the transcription factor and enhanced transcription (Dinkova-Kostova et al., Proc. Natl. Acad. Sci. USA 2002, 99:11908-11913; Itoh et al., Genes Dev. 1999, 13:76-86; McMahon et al., J Biol Chem 2003, 278:21592-21600; Zhang et al., Mol Cell Biol. 2003, 23:8137-8151). Several efforts to define the full gene list regulated by ARE enhancer elements have been made. These include comparative profiling of small molecules inducers of the pathway in wild-type and Nrf2 (−/−) mouse, such as the isothiocyanates phenyl isothiocyanate (PEITC) (Hu et al., Life Sci. 2006, 79:1944-1955) and sulforaphane (Hu et al., Cancer Lett 2006, 243:170-192; Thimmulappa et al., Cancer Res 2002, 62:5196-5203) as well as the green tea extract (−)-epigallocatechin-3-gallate (EGCG) (Shen et al., Pharm. Res. 2005, 22:1805-1820) and ³H-1,2-dithiole-3-thione (D3T) (Kwak et al., J. Biol. Chem. 2003, 278:8135-8145). Consensus genes and gene families commonly induced in these analyses include ferritin, heme-oxygenease 1 (HO-1), NAD(P)H:quinine oxidoreductases (NQO1), glutamate cysteine ligase catalytic (GCLC) and glutamate cysteine ligase modifier (GCLM) subunits, and glutathione-S-transferases (GSTs).

Disruption of the interaction between Keap1 and Nrf2 results in activation of the Keap1-Nrf2-ARE pathway. The genetic knockdown of the Keap1 protein in human keratinocyte cell line provides evidence that relief of Nrf2 repression results in transcription of ARE-dependent genes (Kwak et al. ibid.). In these studies, a 70% reduction in Keap1 mRNA and a corresponding reduction in Keap1 protein levels followed transfection of anti-Keap1 siRNA. Within 24 hours of transfection, Nrf2 protein levels and transcription of an ARE-luciferase reporter gene construct were increased. A 3-fold increase in NQO1, 5-fold increase in GCLC, and a 2.5 fold increase in GCLM within this cell line verify that disruption of Keap1's capacity to regulate Nrf2 protein levels result in increases in ARE-dependent gene transcription (Kwak et al. ibid).

Evidence has emerged over the last decade indicating that activation of the antioxidant response element may be beneficial to the whole organism. Nrf2 mediated activation and resultant ARE-regulated gene induction results in improved outcomes in several animal models of disease, including cancer (Iida et al., Cancer Res. 2004, 64:6424-6431; Ramos-Gomez et al., Proc. Natl. Acad. Sci. USA 2001, 98:3410-3415; Xu et al., Cancer Res. 2006, 66:8293-8296; Yates et al., Cancer Res. 2006, 66:2488-2494), Huntington's (Shih et al., J. Biol. Chem. 2005, 280:22925-22936), Parkinson's (Burton et al., Neurotoxicology 2006), stroke (Satoh et al., Proc. Natl. Acad. Sci. USA 2006, 103:768-773; Shih et al., J. Neurosci. 2005, 25:10321-10335; Zhao et al., Neurosci. Lett. 2006, 393:108-112), and emphysema (Ishii et al., J. Immunol. 2005, 175:6968-6975). We propose that activation of the antioxidant response element, mediated through the targeted disruption of the Keap1 containing ubiquitination complex or the interaction between Keap1 and Nrf2 may be the long sought after antioxidant therapy for the various diseases caused or exacerbated by oxidative stress. However, the development of safe and effective small molecule activators of the Keap1-Nrf2-ARE pathway remains a challenge. Thus, there is a need for a method for identifying modulators of the Keap1-Nrf2-ARE pathway, in particular modulators that inhibit binding of Nrf2 to the Keap1 protein and thus, activate protective phase 2 oxidative enzymes.

### BRIEF SUMMARY OF THE INVENTION

The present invention provides a method for identifying modulators of the (Keap1 protein, nuclear factor erythroid 2, antioxidant response element pathway (Keap1-Nrf2-ARE pathway). In particular, provided is an assay that identifies molecules that inhibit the binding of a labeled Nrf2 peptide with the kelch domain of the Keap1 protein. Molecules that inhibit binding can be activators of the Keap1-Nrf2-ARE pathway. Activation of the Keap1-Nrf2-ARE pathway results in an increased accumulation of Nrf2 and the subsequent induction of protective enzymes, for example, the phase 2 detoxification enzymes. Activators of the Keap1-Nrf2-ARE pathway are useful for combating oxidative stress-related disorders, such as those associated with cancer, emphysema, Huntington's disease, light-induced retinal damage, and stroke.

Therefore, in one aspect, a method is provided for identifying an agent that activates the Keap1-Nrf2-ARE pathway, which comprises providing a mixture including a Keap1 protein or Keap1-kelch domain polypeptide and an Nrf2 peptide that is capable of binding the kelch domain of the Keap1 protein; adding an analyte to be evaluated for its ability to activate the Nrf2 system to the mixture; and, determining the amount of Nrf2 peptide bound to the Keap1 protein or Keap1-kelch domain polypeptide, wherein a decrease in the amount of the Nrf2 peptide bound to the Keap1 protein or Keap1-kelch domain polypeptide compared to the amount of the Nrf2 peptide bound to the Keap1 protein or Keap1-kelch domain polypeptide in the absence of the analyte indicates that the analyte activates the Keap1-Nrf2-ARE pathway.

In particular aspects of the method, the Nrf2 peptide is radiolabeled or is labeled with one member of a donor-acceptor fluorophore pair and the Keap1 protein or Keap1-kelch domain polypeptide is labeled with the other member of the fluorophore pair and fluorescence resonance energy transfer (FRET) or time-resolved FRET (TR-FRET) is measured to determine the amount of Nrf2 peptide bound to the Keap1 protein or kelch domain wherein a decrease in fluorescence over time from the acceptor fluorophore in the presence of the analyte and/or an increase in fluorescence over time from the donor fluorophore in the presence of the analyte indicates that the analyte activates the Keap1-Nrf2-ARE pathway. In currently preferred aspects, the Nrf2 peptide comprises an amino acid sequence selected from the group consisting of SEQ ID NO:1 and SEQ ID NO:2 and in further still aspects, the Keap1 protein or Keap1-kelch domain polypeptide is a fusion protein.

In further aspects, Nrf2 peptide is replaced with an Nrf1 peptide or a Pgam5 peptide. In currently preferred aspects, the Nrf1 peptide comprises the amino acid sequence of SEQ ID NO:7 and the Pgam5 peptide comprises the amino acid sequence of SEQ ID NO:8.

The above method can be performed using a heterogeneous format wherein the Keap1 protein or Keap1-kelch domain polypeptide is immobilized to the surface of a solid support. In particular aspects, the Keap1 protein or kelch domain has a polyhistidine tag. In further aspects, the Keap1 protein or Keap1-kelch domain polypeptide having the polyhistidine tag is immobilized to the surface of the solid support via divalent metal ions or the Keap1 protein or Keap1-kelch domain polypeptide having the polyhistidine tag is immobilized to the surface of the solid support using antibodies specific for the polyhistidine tag which have been immobilized to the surface of the solid support.

The above method can also be performed using a homogeneous format wherein the Keap1 protein or Keap1-kelch domain polypeptide is labeled with one member of a donor-acceptor fluorophore pair and the Nrf2 peptide labeled with the other member of the fluorophore pair and fluorescence resonance energy transfer (FRET) or time-resolved FRET (TR-FRET) is measured to determine the amount of Nrf2 peptide bound to the Keap1 protein or kelch domain wherein a decrease in fluorescence over time from the acceptor fluorophore in the presence of the analyte and/or an increase in fluorescence over time from the donor fluorophore in the presence of the analyte indicates that the analyte activates the Keap1-Nrf2-ARE pathway.

Further provided is a method or system for identifying an analyte that is an activator of the Keap1-Nrf2-ARE pathway, which comprises providing a first assay wherein a mixture of a Keap1 protein or Keap1-kelch domain polypeptide having a polyhistidine tag bound to a labeled Nrf2 peptide is immobilized to the surface of a first solid support via divalent metal ions and a second assay wherein a detectable protein having a polyhistidine tag is immobilized to the surface of a second solid support via divalent metal ions; adding an analyte to be evaluated for ability to activate the Nrf2 system to the first assay and the second assay; and determining the amount of the Nrf2 peptide bound to the Keap1 protein or Keap1-kelch domain polypeptide in the first assay in the presence of the analyte and the amount of detectable protein immobilized to the second solid support in the presence of the analyte, wherein a decrease in the amount of the Nrf2 peptide bound to the Keap1 protein or Keap1-kelch domain polypeptide compared to the amount bound to the Keap1 protein or Keap1-kelch domain polypeptide in the absence of the analyte and no detectable change in the amount of detectable protein immobilized to the surface of the second support indicates that the analyte is an activator of the Keap1-Nrf2-ARE pathway.

In a further aspect, the method includes a third assay in which the Keap1 protein or Keap1-kelch domain polypeptide having the polyhistidine tag bound to the labeled Nrf2 peptide is immobilized to the surface of a third solid support using antibodies specific for the polyhistidine tag wherein the antibodies have been immobilized to the surface of the third solid support; adding the analyte to be evaluated for ability to activate the Keap1-Nrf2-ARE pathway to the third assay; and determining the amount of the Nrf2 peptide bound to the Keap1 protein or Keap1-kelch domain polypeptide in the third assay in the presence of the analyte, wherein a decrease in the amount of the Nrf2 peptide bound to the Keap1 protein or Keap1-kelch domain polypeptide compared to the amount bound to the Keap1 protein or Keap1-kelch domain polypeptide in the absence of the analyte in the first and third assays and no detectable change in the amount of detectable protein immobilized to the surface of the second support indicates that the analyte is an activator of the Keap1-Nrf2-ARE pathway.

In particular aspects of the method, the Nrf2 peptide is radiolabeled or is labeled with one member of a donor-acceptor fluorophore pair and the Keap1 protein or Keap1-kelch domain polypeptide is labeled with the other member of the fluorophore pair and fluorescence resonance energy transfer (FRET) or time-resolved FRET (TR-FRET) is measured to determine the amount of Nrf2 peptide bound to the Keap1 protein or kelch domain wherein a decrease in fluorescence over time from the acceptor fluorophore in the presence of the analyte and/or an increase in fluorescence over time from the donor fluorophore in the presence of the analyte indicates that the analyte activates the Keap1-Nrf2-ARE pathway. In currently preferred aspects, the Nrf2 peptide comprises an amino acid sequence selected from the group consisting of SEQ ID NO:1 and SEQ ID NO:2 and in further still aspects, the Keap1 protein or Keap1-kelch domain polypeptide is a fusion protein.

In further aspects, Nrf2 peptide is replaced with an Nrf1 peptide or a Pgam5 peptide. In currently preferred aspects, the Nrf1 peptide comprises the amino acid sequence of SEQ ID NO:7 and the Pgam5 peptide comprises the amino acid sequence of SEQ ID NO:8.

In further aspects, the detectable protein is labeled with a fluorophore or has a detectable activity, for example, green fluorescence protein.

Further still is provided a homogenous method for identifying an agent that activates the Keap1-Nrf2-ARE pathway, which comprises providing a mixture that includes a Keap1 protein or Keap1-kelch domain polypeptide labeled with one member of a donor-acceptor fluorophore pair and a Nrf2 peptide that is capable of binding the Keap1 protein or Keap1-kelch domain polypeptide labeled with the other member of the donor-acceptor pair, wherein the acceptor fluorophore produces a detectable fluorescence when the Keap1 protein or Keap1-kelch domain polypeptide is bound to the Nrf2 peptide; adding an analyte to be evaluated for its ability to activate the Keap1-Nrf2-ARE pathway to the mixture; and measuring the amount of the detectable fluorescence from the acceptor fluorophore over time wherein a decrease in the amount of detectable fluorescence over time from the acceptor fluorophore in the presence of the analyte indicates that the analyte activates the Keap1-Nrf2-ARE pathway. In particular aspects, the donor fluorophore produces a second detectable fluorescence, which increases over time in the presence of an analyte when the analyte is an activator of the Keap1-Nrf2-ARE pathway. In currently preferred aspects, the Nrf2 peptide comprises an amino acid sequence selected from the group consisting of SEQ ID NO: 1 and SEQ ID NO:2 and in further still aspects, the Keap1 protein or Keap1-kelch domain polypeptide is a fusion protein.

In further aspects, Nrf2 peptide is replaced with an Nrf1 peptide or a Pgam5 peptide. In currently preferred aspects, the Nrf1 peptide comprises the amino acid sequence of SEQ ID NO:7 and the Pgam5 peptide comprises the amino acid sequence of SEQ ID NO:8.

In further aspects, the donor fluorophore includes a lanthanide and time-resolved FRET (TR-FRET) is measured to determine the amount of Nrf2 peptide bound to the Keap1 protein or kelch domain wherein a decrease in fluorescence over time from the acceptor fluorophore in the presence of the analyte and/or an increase in fluorescence over time from the donor fluorophore in the presence of the analyte indicates that the analyte activates the Keap1-Nrf2-ARE pathway. In particular aspects, lanthanide is Eu3⁺ or the donor fluorophore is Europium cryptate.

### BRIEF DESCRIPTION OF THE DRAWINGS

FIG. 1A shows a dose response curve for an ion-based assay in which a mixture containing His-tagged Keap1-kelch domain polypeptide and ³H-labeled Peptide 2 was incubated with analyte A. The His-tagged Keap1-kelch domain polypeptide was immobilized to the surface of wells coated with divalent nickel ions.

FIG. 1B shows a dose response curve for an ion-based assay in which a mixture containing His-tagged Keap1-kelch domain polypeptide and ³H-labeled Peptide 2 was incubated with unlabeled peptide 2. The His-tagged Keap1-kelch domain polypeptide was immobilized to the surface of wells coated with divalent nickel ions.

FIG. 2 shows a dose response curve for an antibody-based counterscreen assay in which a mixture containing His-tagged Keap1-kelch domain polypeptide and ³H-labeled Peptide 2 was incubated with various analytes, including analyte A and unlabeled peptide 2. The His-tagged Keap1-kelch domain polypeptide was immobilized to the wells of a plate coated with anti-mouse IgG using mouse-derived anti-His tag antibodies. ▾-Analyte A; □-Analyte B; ♦-Ananlyte C; Δ-unlabeled Peptide 2.

FIG. 3 FIG. 2 shows a His-GFP binding assay in which His-tagged GFP, which had been immobilized to the surface of wells coated with divalent nickel ions, was incubated with various analytes, including analyte A and unlabeled peptide 2. The His-tagged Keap1-kelch domain polypeptide was immobilized to the wells of a plate coated with anti-mouse IgG using mouse-derived anti-His tag antibodies. ▾-Analyte A; □-Analyte B; ♦-Analyte C; Δ-unlabeled Peptide 2.

### DETAILED DESCRIPTION OF THE INVENTION

The present invention provides a method for identifying analytes that disrupt the interaction of Nrf2 transcription factor with the kelch domain of the Keap1 protein to form an Nrf2-Keap1 complex. The method provides an assay in which a mixture comprising a peptide substrate corresponding to the portion of the Nrf2 transcription factor that binds to the kelch domain of the Keap1 protein and either the Keap1 protein or a polypeptide comprising the kelch domain of the Keap1 protein (Keap1-kelch domain polypeptide) is incubated with an analyte being tested for its ability to disrupt binding of the peptide to the kelch domain. Analytes identified using the assay may mimic intracellular disruption of the Nrf2-Keap1 complex, which leads to increased accumulation of Nrf2 transcription factor and the subsequent induction of protective enzymes, for example, the phase 2 detoxification enzymes. Analytes identified in the assay as inhibitors of the formation of the Nrf2-Keap1 complex and thus activators of the Nrf2 system are useful for combating oxidative stress-related disorders associated with cancers, emphysemia, Huntington's disease, light-induced retinal damage, cardiovascular disease, Parkinson's disease, Alzheimer's disease, and stroke.

There are many protein:protein interactions required for ARE-mediated gene induction to occur, such as Nrf2 with small Maf proteins and Nrf2 association with kinases. While any of these points of interaction could be potential sites of modulation of the Keap1-Nrf2 system, detailed structural analysis of the Cul3:Keap1 and Keap1:Nrf2 interactions exist and warrant consideration as targets for small molecule disruptors of these interactions. Molecules that act by disruption of protein:protein interactions in the Keap1-Nrf2 system will provide a novel means to modulate the system without the liabilities associated with many of the known compounds that activate this pathway.

### Keap1:Nrf2 Interaction

The Keap1:Nrf2 interface is another site for targeted disruption using small molecules. High resolution structural information has been gathered for the Keap1 kelch domain (Li et al., J. Biol. Chem. 2004, 279:54750-54758) and its interaction with small portions of Nrf2 (Lo et al., EMBO J. 2006, 25:3605-3617; Padmanabhan et al., Mol. Cell. 2006, 21:689-700). These data coupled with mutagenesis data provide a wealth of information on specific molecular contacts between Keap1 and Nrf2. This information may be exploited by computer aided drug design to identify compound that can be tested for the ability to dissociate Nrf2 and Keap1 in order to enhance the transcription of ARE containing genes.

The three dimensional structure of the Keap1 kelch domain was determined by X-ray crystallography by two different groups (Li et al., J. Biol. Chem. 2004, 279:54750-54758; Lo et al., EMBO J 2006, 25:3605-3617; Padmanabhan et al., Mol. Cell. 2006, 21:689-700). The kelch domain is a six-bladed β-propeller with each blade consisting of four β-sheets and corresponding to a single kelch repeat motif. The central core of the structure contains a water filled channel. The structure determined for the human kelch domain consisted solely of the kelch domain (Li et al., J. Biol. Chem. 2004, 279:54750-54758), whereas that determined for the mouse contains both the kelch domain and the C-terminal region of 15 amino acids Padmanabhan et al., Mol. Cell. 2006, 21:689-700). As expected, the human and mouse structures are nearly identical with the exception that the mouse structure reveals that the C-terminal region contributes to the folding of the kelch domain. The significance of this contribution is underscored by mutagenesis data indicating the critical role of specific amino acids in the C-terminal region in enabling Keap1 repression of Nrf2 (Padmanabhan et al., Mol. Cell. 2006, 21:689-700).

The understanding of the molecular interactions between Keap1 and Nrf2 increased greatly with the determination of the three dimensional structure of the mouse Keap1 kelch domain bound to Nrf2 derived peptides of either 9 residues (aa 76-84) or 16 residues (aa 74 to 89) (Padmanabhan et al., Mol. Cell. 2006, 21:689-700) and of the human Keap1 kelch domain bound to a 16 residue Nrf2 derived peptide (aa 69 to 84) (Lo et al., EMBO J 2006, 25:3605-3617). The Nrf2 derived peptides adopt a tight type 1 β-turn when bound to Keap1, with the highly conserved sequence DxETGE (corresponding to Nrf2 amino acids 77-82) comprising the tip of the hairpin (32; Padmanabhan et al., Mol. Cell. 2006, 21:689-700). Inter-molecular contacts are made between the side chains of Nrf2 amino acid Glu-79 and Keap1 amino acid Ser508, Arg415, and Arg483 (Lo et al., EMBO J 2006, 25:3605-3617; Padmanabhan et al., Mol. Cell. 2006, 21:689-700). A second set of interactions occurs between the side chain of Nrf2 Glu82 and Keap1 Arg380 and Ser363. There are additional contacts made between the peptide backbone of Nrf2 and Keap1 residues. Most of the contacts that occur in the crystal structure have been further tested by mutagenesis to establish whether they occur in the complex environment of the cell (32). As expected, there is a loss of Nrf2 repression when the key contacts in Keap1, Arg380, Arg415 and Arg483, are individually mutated to Ala (Lo et al., EMBO J 2006, 25:3605-3617). Additional Keap1 amino acid residues that contact Nrf2 and when mutated have a diminished ability to repress Nrf2 include Asn380, Asn382 and Tyr334 (Lo et al., EMBO J 2006, 25:3605-3617). There are a number of molecular contacts, Keap1 residues Ser363, Ser508, Gln530, Ser555, and Ser602 that appear to be somewhat dispensable, in that there is a lack of a readily discernable defect in Nrf2 repression when these residues are mutated (Lo et al., EMBO J 2006, 25:3605-3617). The structure of the Nrf2 peptide which inserts into the Keap1 substrate binding site is stabilized by intramolecular interactions (Lo et al., EMBO J 2006, 25:3605-3617; Padmanabhan et al., Mol. Cell. 2006, 21:689-700). Phosphorylation of Nrf2 residue Thr80 or mutation of this amino acid to either Asp or Glu, results in an apparent destabilization of the β-turn and reduced affinity for Keap1 (Lo et al., EMBO J 2006, 25:3605-3617). Phosphorylation of Nrf2 at Thr80 and other key residues is a likely mechanism for the modulation of Nrf2 activity by stress responsive kinases. Taken together, the three dimensional crystal structure coupled with mutagenesis studies has provided a detailed understanding of the Keap1:Nrf2 interaction that has the potential to be exploited by drug discovery efforts.

The stoichiometry of Keap1 and Nrf2 remains unresolved, with conflicting data from two different groups. Keap1 is known to form a homodimer through the BTB domain; therefore the crystal structure of the kelch domain alone complexed with an Nrf2 peptide sheds little light on the stoichiometry. The Yamamoto lab has proposed that the Keap1 dimer binds to a single Nrf2 molecule (Tong et al., Mol. Cell. Biol. 2006, 26:2887-2900). The 2:1 stoichiometry proposed by Yamamoto and colleagues is based primarily on a combination of NMR spectra, isothermal calorimetry and biochemical data. In their model, the Keap1 substrate binding pocket from one subunit is occupied by the Nhe2 domain ETGE motif, whereas, the substrate binding pocket of the second subunit of the dimer is occupied by the DLG motif, also located in the Neh2 domain. The binding of a single Nrf2 molecule to a Keap1 dimer is proposed to effectively position an alpha-helix containing Lys residues for ubiquitination (Tong et al., Mol. Cell. Biol. 2006, 26:2887-2900; McMahon et al., J. Biol. Chem. 2006, 281:24756-24768). The affinity of the ETGE motif for Keap1 is approximately two orders of magnitude higher than that for the DLG motif (Tong et al., Mol. Cell. Biol. 2006, 26:2887-2900; McMahon et al., J. Biol. Chem. 2006, 281:24756-24768). Therefore, it should be feasible to identify small molecules that disrupt the low affinity interaction between the Keap1 substrate binding pocket and the DLG motif. The disruption of the low affinity interaction between Keap1 and Nrf2 is unlikely to result in dissociation of Nrf2 from Keap1, due to the high affinity interaction with the ETGE motif. However, disruption of the low affinity interaction would be predicted to block the ubiquitination of Nrf2 and therefore may lead to activation of the system.

In contrast, a model with 1:1 Keap1:Nrf2 stoichiometry has been proposed by Hannink and colleagues (Lo et al., EMBO J 2006, 25:3605-3617). This model is based upon biochemical and molecular biology experiments. Utilizing differentially tagged Nrf2 and Keap1, this group designed an elegant experiment to examine the association of Keap1 and Nrf2 in the cellular environment. The Neh2 domain of Nrf2 was tagged with either a Heme-Agglutinin (HA) tag or a Gal-4 tag, with the two versions being co-expressed in either the presence or absence of Keap1. In the absence of Keap1, immunoprecipitation with antibody to the HA tag did not pull down Gal-4 tagged Neh2. In contrast, when Keap1 was co-expressed in the cell the Gal-4 tagged Neh2 domain was immunoprecipitated with antibody to the HA tag, providing evidence that Keap1 linked the two different versions of the Neh2 domain due to the ability of a Keap1 dimer to bind two molecules of Nrf2 (Lo et al., EMBO J 2006, 25:3605-3617). Additional three dimensional structural data will help resolve whether the Keap1 homodimer binds a single Nrf2 molecule or whether the alternative hypothesis of a 1:1 stoichiometry is correct, in which case the low affinity interaction between the DLG motif and Keap1 may be a cryptic function unveiled by the nature of the experiments conducted.

Modulation of Nrf2's activity through disruption of Keap1 protein:protein interactions may have advantages over present means of activating the ARE pathway. Several known small molecule activators of the ARE system transducer their effects through electrophilic attack on the available thiol residues of Keap1, a concept originally proposed and demonstrated by Dinkova-Kostova et al. (Dinkova-Kostova et al., Proc. Natl. Acad. Sci. USA 2001, 98:3404-3409). Recent proteomic studies utilizing mass spectroscopy have revealed that alkylation of certain cysteine residues contained within the Keap1 BACK domain by compounds such as N-iodoacetyl-N-biotinylhexylenediamine (IAB) result in Nrf2 translocation to the nucleus and ARE-mediated gene expression. This is not a general phenomenon, however, as not all electrophiles examined regulated ARE activity. 1-biotinamido-4-(4′-[maleimidoethyl-cyclohexane]-carboxamido)butane (BMCC) treatment resulted in alkylation of Keap1 at sites outside of the linker region, and had no impact on ARE-dependent gene expression (Hong et al., J. Biol. Chem. 2005, 280:31768-3177). Thus, the site of adduction and resulting ARE induction, if any, appears highly dependent on the chemistry of the electrophile used as well as its site of interaction within Keap1. Sulforaphane, an isothiocyanate and prototypical inducer of the ARE, targets cysteine residues within the BTB and kelch domains along with the BACK domain (Hong et al., J. Biol. Chem. 2005, 280:31768-3177). Incubation of cells with sulforaphane also results in the production of a high molecular weight Keap1 complex, which was subsequently identified as polyubiquitinated Keap1. From this observation, it has been proposed that sulforaphane may not exert its effects by modulating the physical interaction between Keap1 and Nrf2, but rather by enabling a transition from Keap1-mediated Nrf2 ubiquitination to Keap1 's autoubiquitination and subsequent degradation (Zhang and Hannink, Mol Cell Biol 2003, 23:8137-8151; Zhang et al., Mol. Cell. Biol. 2004, 24:10941-10953; Hong et al., J. Biol. Chem. 2005, 280:31768-3177). These electrophiles serve as useful biological tools to explore the mechanisms regulating ARE-dependent gene expression and may be clinically useful in acute disease paradigms. Their clinical utility in chronic disease, such as neurodegenerative diseases, may be limited due to safety concerns arising from their nonspecific alkylation of cellular proteins.

### Modulation of E3 Ligase Protein:Protein Interactions

Recently, successful efforts to modulate protein degradation by the ubiquitin-proteosome pathway have focused on disruption of the protein:protein interactions within the ubiquitin conjugating complex or between the complex and its substrate. Using high-throughput screening methods, scientists at Roche were able to identify the nutlin class of compounds and demonstrate, through crystallographic analysis, that the compounds bound the p53 binding region of the mouse double minute 2 (Mdm2) protein, a RING type E3 ubiquitin ligase. This competitive inhibition at the p53 site results in the induced expression of the p21 gene, cell cycle arrest in the p53 containing cell lines examined, and enhanced cytotoxicity to p53 containing cells. In rodent studies, these small molecules reduce tumor volume, indicating that the disruption of the E3 ligase-transcription factor complex results in meaningful in vivo functional outcomes (Vassilev et al., Science 2004, 303:844-848). The MI-17 (Ding et al., J. Am. Chem. Soc. 2005, 127:10130-10131) series and second generation spiro-oxindoles (Ding et al., J. Med. Chem. 2006, 49:3432-3435), the HL198 related series (Yang et al., Cancer Cell 2005, 7:547-559), and Reactivation of p53 and Induction of Tumor cell Apoptosis (RITA; (Issaeva et al., Nat. Med. 2004, 10:1321-1328)) were also shown to disrupt the interaction between Hdm2/Mdm2 and p53. All of these molecules bind to the hydrophobic p53 binding site of H/Mdm2 (Vassilev et al., Science 2004, 303:844-848; Ding et al., J. Am. Chem. Soc. 2005, 127:10130-10131; Ding et al., J. Med. Chem. 2006, 49:3432-3435; Yang et al., Cancer Cell 2005, 7:547-559), except RITA which binds directly to p53 (Issaeva et al., Nat. Med. 2004, 10:1321-1328). In many ways, the Keap1-Nrf2 system is analogous to the H/Mdm2-p53 system. Both transcription factors, p53 and Nrf2, are targeted for proteosomal degradation by their respective E3 ubiquitin ligases, Mdm2 and Keap1. Further, Mdm2 is a component of a Cul4A-DDB1 complex (Banks et al., Cell Cycle 2006, 5:1719-1729). Analogous to p53's interaction with H/Mdm2, the Nrf2 binding site located on the Keap1 kelch domain is a defined pocket on the surface of the protein (Klebanoff, J. Leukoc. Biol. 2005, 77:598-625)). The successful identification of the Mdm2-p53 disrupting compounds provides precedence for the idea that small molecule protein:protein disruptors of the Keap1-containing ubiquitination complex and it's interactions with Nrf2 will be realized.

Evidence has emerged over the last decade indicating that activation of the antioxidant response element may be beneficial to the whole organism. Nrf2 mediated activation and resultant ARE-regulated gene induction results in improved outcomes in several animal models of disease, including (Iida et al., Cancer Res. 2004, 64:6424-6431; Ramos-Gomez et al., Proc. Natl. Acad. Sci. USA 2001, 98:3410-3415; Xu et al., Cancer Res. 2006, 66:8293-8296; Yates et al., Cancer Res. 2006, 66:2488-2494), Huntington's (Shih et al., J. Biol. Chem. 2005, 280:22925-22936), Parkinson's (Burton et al., Neurotoxicology 2006), stroke (Satoh et al., Proc. Natl. Acad. Sci. USA 2006, 103:768-773; Shih et al., J. Neurosci. 2005, 25:10321-10335; Zhao et al., Neurosci. Lett. 2006, 393:108-112), and emphysema (Ishii et al., J. Immunol. 2005, 175:6968-6975). We propose that activation of the antioxidant response element, mediated through the targeted disruption of the Keap1 containing ubiquitination complex or the interaction between Keap1 and Nrf2 may be the long sought after antioxidant therapy for the various diseases caused or exacerbated by oxidative stress. However, the development of safe and effective small molecule activators of the Keap1-Nrf2-ARE pathway remains a challenge. The present invention provides a method for identifying analytes that disrupt the interaction of Nrf2 transcription factor with the kelch domain of the Keap1 protein to form an Nrf2-Keap1 complex and thus provides a method for identifying activators of the Keap1-Nrf2-ARE pathway.

In general, there are two classes of assay formats for methods that can be used for identifying inhibitors of Nrf2 binding to the kelch domain of the Keap1 protein depending upon whether the assay requires the separation of bound species from unbound species. In assays that have a heterogeneous format, a separation or isolation step is required to remove bound material from unbound material. In contrast, in assays that have a homogeneous format, removal of bound species from unbound species is unnecessary. Because homogeneous assays lack a separation step, and are more easily automated, they can be more desirable than heterogeneous assays in applications that entail the screening of large numbers of analytes. The method for identifying analytes that disrupt the interaction of Nrf2 transcription factor with the kelch domain of the Keap1 protein includes assays that have a heterogeneous format and assays that have a homogeneous format. For particular assays, the Keap1-kelch domain polypeptide comprises polypeptide in which the kelch domain is fused to a heterologous protein or polypeptide.

### Heterogeneous Format Assays

For assays having a heterogeneous format, the Keap1 protein or the Keap1-kelch domain polypeptide is immobilized onto the surface of a solid support. The immobilized protein or polypeptide is then incubated with a mixture comprising a labeled Nrf2 peptide comprising the amino acid sequence of the Nrf2 transcription factor that binds the kelch domain of the Keap1 protein. In currently preferred embodiments, the Nrf2 peptide comprises at least the 14 amino acids shown in SEQ ID NOs:1 and 2. The analyte being tested for ability to interfere with the binding of the labeled Nrf2 peptide to the kelch domain is added to the mixture at the same time the labeled peptide is added to the mixture or at a time after the labeled peptide had been added to the mixture. Afterwards, the mixture is removed and the amount of labeled Nrf2 peptide remaining bound to the Keap1 protein or Keap1-kelch domain polypeptide is determined. If the analyte disrupts the binding of the Nrf2 peptide to the kelch domain, the amount of labeled Nrf2 peptide bound to the kelch domain is diminished compared to the negative control. Preferably, the assay includes a negative control that does not include the analyte and a positive control that includes a molecule that competes with the labeled Nrf2 peptide for binding to the kelch domain. A molecule suitable as a positive control is the Nrf2 peptide not labeled.

In a general format for a heterogeneous assay, the Keap1 protein or Keap1-kelch domain polypeptide is immobilized to the surface of the solid support by providing a Keap1 protein or Keap1-kelch domain polypeptide labeled with a polyhistidine sequence (His-tag) at the amino or carboxy terminus of the protein. The His-tagged protein or polypeptide is then immobilized on a divalent metal ion solid support, in general, the divalent metal ion is usually nickel or copper. Example 4 provides an example wherein a His-tagged Keap1 protein was immobilized in the wells of multiwell assay plates which had been coated with divalent nickel ions. Alternatively, the His-tagged Keap1 protein or Keap1-kelch domain polypeptide can be immobilized on the solid support using antibodies specific for the His tag. For example, the solid support can be coated with anti-mouse IgG antibodies which bind a mouse anti-His tag antibody bound to the His-tagged Keap1 protein or Keap1-kelch domain polypeptide. Example 5 provides an example wherein the His-tagged Keap1 protein was bound to a mouse anti-His tag antibody which was in turn immobilized to the surface of the wells of plates which had been coated with anti-mouse IgG antibodies. Alternative means for immobilizing the Keap1 protein or Keap1-kelch domain polypeptide to the surface of the solid support are well known in the art and, include but are not limited to, using antibodies that bind directly to the Keap1 protein or Keap1-kelch domain polypeptide and which have been immobilized to the surface of the solid support, providing the Keap1 protein or Keap1-kelch domain polypeptide tagged with glutathione-S-transferase (GST) and immobilizing the fusion protein to a solid support coated with glutathione, labeling the keap1 protein or keap1-kelch domain polypeptide with biotin and immobilizing the labeled Keap1 protein or Keap1-kelch domain polypeptide to a solid support coated with streptavidin, and covalently linking the Keap1 protein or Keap1-kelch domain polypeptide directly to the surface of the solid support. In a currently preferred embodiment, the Keap1-kelch domain polypeptide comprises amino acid residues 322 to 609 of the human Keap1 protein. In particular embodiments, the Keap1 protein or Keap1-kelch domain polypeptide can be fused to a heterologous protein or polypeptide.

In the general format, the Nrf2 peptide substrate is labeled with a detectable label, for example, a radiolabel (for example, tritium), a fluorescent label, an antibody, biotin, lanthanide ion complex, or the like. The amount of labeled peptide dissociated from the Keap1 protein or Keap1-kelch domain polypeptide immobilized on the solid support in the presence of an analyte is then determined. In particular formats, the Keap1 protein or Keap1-kelch domain polypeptide is labeled with a detectable label that is distinguishable from the label on the Nrf2 peptide substrate. In further formats, fluorescence resonance energy transfer (FRET) is used to measure the ability of an analyte to interfere with the binding of the labeled Nrf2 peptide substrate from immobilized Keap1 protein or Keap1-kelch domain polypeptide. In a FRET format, the Nrf2 peptide substrate is labeled with a donor fluorophore and the immobilized Keap1 protein or Keap1-kelch domain polypeptide is labeled with an acceptor fluorophore or vice versa. The amount of labeled peptide dissociated from the Keap1 protein or Keap1-kelch domain polypeptide immobilized on the solid support in the presence of an analyte is then determined by measuring the decrease in fluorescence from the acceptor fluorophore over time in the presence of the analyte. In some cases, there is also an increase in fluorescence from the donor fluorophore. In further formats, FRET is combined with time-resolved fluorescence (TR-FRET) or variations thereof. For high throughput screening, it is desirable that the Nrf2 peptide be labeled with a fluorescent label or that the assay is performed using a FRET or TR-FRET format or variations thereof.

The above format in which a His-tagged Keap1 or kelch polypeptide is immobilized to a solid support via divalent metal ions (ion-based assay) is desirable because of the ability to immobilize a greater number of His-tagged protein or polypeptide per unit area of the solid support than using antibodies to immobilize the His-tagged protein or polypeptide (antibody-based assay). However, as shown in the Examples, analytes can either compete with the His-tagged Keap1 or Keap1-kelch domain polypeptide for binding to divalent metal ions or destabilize the binding of the His-tagged Keap1 protein or Keap1-kelch domain polypeptide to the divalent metal ions.

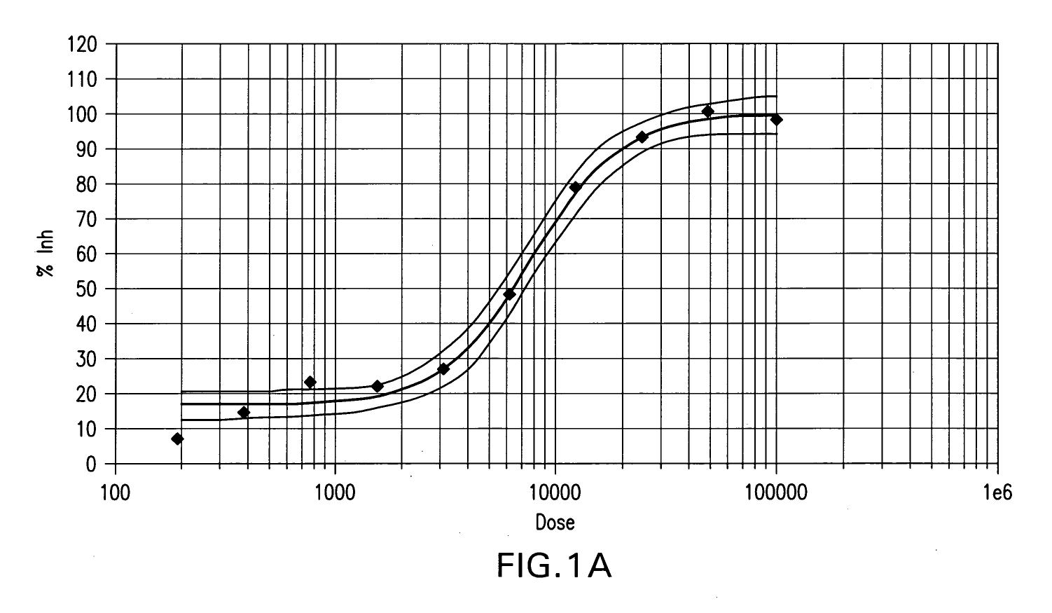

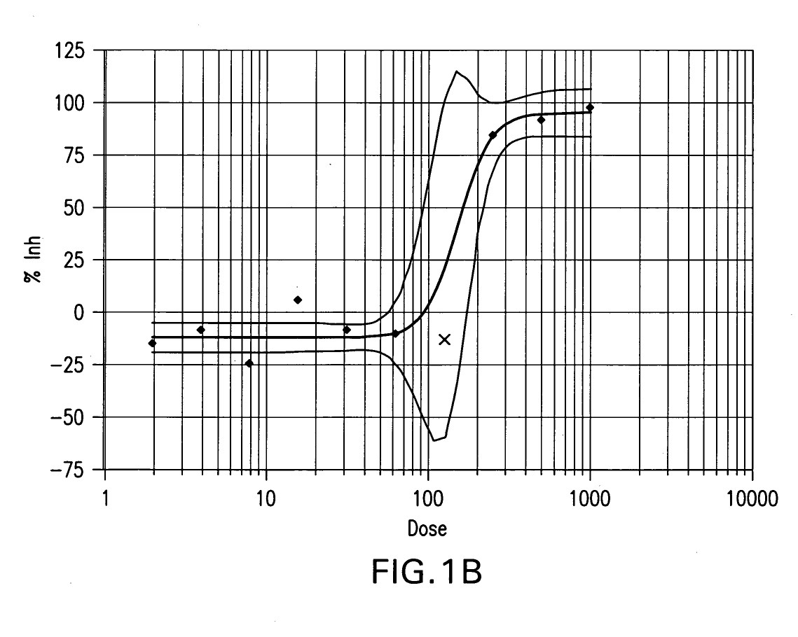

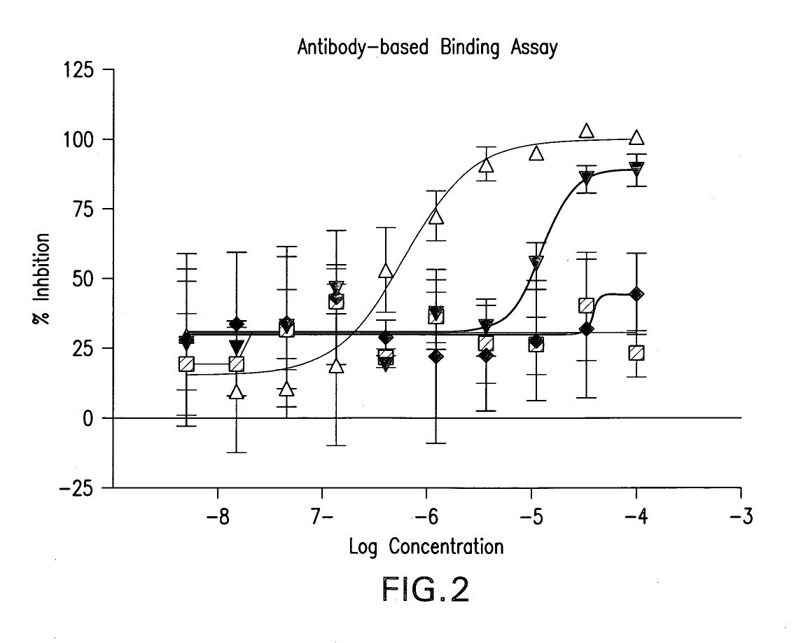

The ion-based assay measures the amount of labeled Nrf2 peptide associated with the solid support (that is, bound to the kelch domain of the Keap1 protein or Keap1-kelch domain polypeptide) in the presence of an analyte and any decrease in the amount of labeled Nrf2 peptide associated with the solid support during the course of the assay indicates that the analyte is a competitor of the labeled peptide for binding to the kelch domain. However, analytes that compete with or destabilize the binding of the His-tagged Keap1 protein or Keap1-kelch domain polypeptide to the divalent metal ions on the surface of the solid support and not binding of the Nrf2 peptide to the kelch domain will also cause a decrease in the amount of labeled peptide associated with the solid support and thus, a “false positive” result. To distinguish analytes that interfere with binding of the labeled Nrf2 peptide with the Keap1 protein or Keap1-kelch domain polypeptide from analytes that produce a false positive result, it is preferable that the assay be performed using the ion-based assay followed by performing an antibody-based assay in which a His-tagged Keap1 or Keap1-kelch domain polypeptide is immobilized to a solid support via antibodies specific for the His tag. As shown in Example 5, analyte A, which had appeared to displace the labeled Nrf2 peptide bound to His-tagged Keap1 immobilized to nickel ion coated plates as shown in FIG. 1A was found using the antibody-based assay to provide inconsistent positive results (FIG. 2) suggesting that the displacement observed in FIG. 1A might be the result of analyte A competing with the His-tagged Keap1 protein for binding to the nickel ion and not the result of the analyte competing with the labeled Nrf2 peptide for binding to the kelch domain of the Keap1 protein.

While the antibody-based assay can in many cases enable identification of analytes that interfere with binding of the Nrf2 peptide to the kelch domain of the Keap1 protein of Keap1-kelch domain polypeptide, the amount of Keap1 protein of Keap1-kelch domain polypeptide immobilized to a unit area of the solid support is less than that which can be immobilized to a solid support via divalent metal ions. Furthermore, for particular analytes the results can be inconsistent or equivocal (for example, analyte A as shown in FIG. 2). Therefore, a counterscreen to the ion-based assay designed to detect analytes that compete with the His-tagged protein or polypeptide for binding to the divalent metal ion attached to the surface of the solid support was developed that measures the ability of an analyte to displace the binding of a His-tagged protein or polypeptide to a divalent metal ion.

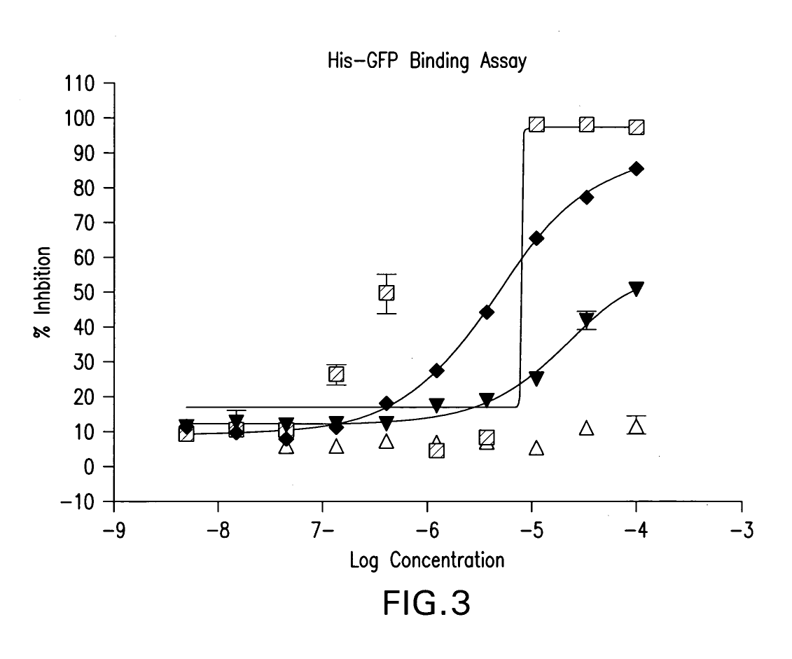

In this assay, a His-tagged protein or polypeptide is immobilized to the surface of a solid support coated with a divalent metal ion. The immobilized His-tagged protein or polypeptide is incubated with the analyte and the amount of His-tagged protein or polypeptide dissociated from the divalent metal ions is measured. An increase in the amount of His-tagged protein or polypeptide displaced from the solid support indicates that the analyte is a competitor of His tag for binding to the divalent metal ion and that the displacement effect observed in the ion-based assay is the result of the analyte competing with the His-tagged Keap1 or Keap1-kelch domain polypeptide with the divalent metal ion and not the analyte competing with the labeled Nrf2 peptide for binding to the kelch domain of the Keap1 protein or the Keap1-kelch domain polypeptide. In general, the His-tagged protein can be any protein that is labeled or has an activity that can be measured. Example 6 provides an example where His-tagged green fluorescence protein (GFP) was immobilized to the surface of multiwell assay plates coated with divalent nickel ions and incubated with various analytes, including analyte A. As shown in FIG. 3, analyte A was able to compete with His-tagged GFP for binding to the divalent nickel ions. The results show that the above counterscreen is important for determining whether the analytes that had been identified in the ion-based assay interfere with binding of the labeled Nrf2 peptide substrate with the kelch domain of the Keap1 protein or Keap1-kelch domain polypeptide, which is desired, or interfere with binding of the Keap1 protein or Keap1-kelch domain polypeptide with the divalent metal ions.

While the ion-based assay can be used to identify analytes that interfere with or disrupt binding of Nrf2 to the kelch domain of the Keap1 protein, it is preferable that the method for identifying such analytes include performing the ion-based assay and either the counterscreen assay (for example, the GFP counterscreen disclosed herein) or the antibody-based assay. In further still aspects of the method for identifying such analytes, the method includes performing the ion-based assay, the counterscreen assay (for example, the GFP counterscreen disclosed herein), and the antibody-based assay. Analytes identified using any combination of heterogeneous format assays may be useful for use in treatments and therapies for oxidative stress-related disorders in an individual where an increase in the accumulation of Nrf2 transcription factor in the cells of the individual effects the subsequent induction of protective enzymes.

### Homogeneous Format Assays

A homogeneous format assay can also be used to identify analytes that interfere with or disrupt binding of Nrf2 to the kelch domain of the Keap1 protein. In general, a FRET or time-resolved FRET (TR-FRET) format or variations thereof is used for identifying analytes that interfere with or disrupt binding of Nrf2 to the kelch domain of the Keap1 protein and; therefore, may be useful in treatments and therapies for oxidative stress-related disorders. A homogenous format assay is particularly suitable for high throughput screening assays.

In a FRET format, the Keap1 protein or Keap1-kelch domain polypeptide is labeled with a fluorphore acceptor and the Nrf2 peptide is labeled with a fluorophore donor or vice versa. The labeled protein or polypeptide and Nrf2 peptide are incubated together for a time sufficient for the Nrf2 protein to bind the kelch domain. When the labeled protein or polypeptide and the Nrf2 peptide are bound, the energy transfer between the donor and acceptor fluorophores can be measured as fluorescence from the acceptor fluorophore. In some cases, there is a decrease in fluorescence from the donor fluorophore. Next, an analyte to be tested is added and the affect on the energy transfer between the donor and acceptor fluorophores is measured. A decrease in fluorescence from the acceptor fluorophore over time indicates that the analyte competes with the labeled Nrf2 peptide for binding to the kelch domain. In some cases there is also an increase in fluorescence from the donor fluorophore which can be measured. Thus, an increase in donor fluorophore fluorescence indicates that the analyte competes with the labeled Nrf2 peptide for binding to the kelch domain. In currently preferred embodiments, the Nrf2 peptide comprises at least the 14 amino acids shown in SEQ ID NOs:1 and 2. In a currently preferred embodiment, the Keap1 protein or Keap1-kelch domain polypeptide comprises amino acid residues 322 to 609 of the human Keap1 protein. In particular embodiments, the Keap1 protein or Keap1-kelch domain polypeptide can be fused to a heterologous protein or polypeptide.

In an TR-FRET format, the Keap1 protein or Keap1-kelch domain polypeptide is labeled with a fluorophore donor, which comprises a lanthanide preferably in complex with a moiety for harvesting light and transferring it to the lanthanide (for example, a chelate or cryptate) and the Nrf2 peptide is labeled with a fluorphore acceptor that is capable of accepting the energy transfer from the lanthanide or vice versa. The labeled protein or polypeptide and Nrf2 peptide are incubated together for a time sufficient for the Nrf2 protein to bind the kelch domain. When the labeled protein or polypeptide and the Nrf2 peptide are bound, the energy transfer between the donor and acceptor fluorophores can be measured as fluorescence from the acceptor fluorophore. Next, an analyte to be tested is added and the energy transfer between the donor and acceptor fluorophores is measured. A decrease in fluorescence from the acceptor fluorophore over time indicates that the analyte competes with the labeled Nrf2 peptide for binding to the kelch domain.

A useful TR-FRET format is called (homogenous time resolved fluorescence or HTRF, a registered trademark of Cisbio International) wherein the lanthanide is Eu³⁺ conjugated to cryptate (trisbypyridine). In an example of an HTRF format, the Keap1 protein or Keap1-kelch domain polypeptide is labeled with Europium cryptate (trisbypyridine in which an Eu³⁺ ion is embedded) and the Nrf2 peptide is labeled with a fluorphore acceptor that is capable of accepting the energy transfer from the Europium cryptate (for example, XL665, a phycobilliprotein from red algae or vice versa). The labeled protein or polypeptide and Nrf2 peptide are incubated together for a time sufficient for the Nrf2 protein to bind the kelch domain. When the labeled protein or polypeptide and the Nrf2 peptide are bound, the energy transfer between the donor and acceptor fluorophores can be measured at 655 nm. Next, an analyte to be tested is added and the energy transfer between the donor and acceptor fluorophores is measured. A decrease in fluorescence at 655 nm over time indicates that the analyte competes with the labeled Nrf2 peptide for binding to the kelch domain and may be useful in treatments and therapies for oxidative stress-related disorders.

FRET has been described in, for example, Wolf et al., Proc. Nat. Acad. Sci. USA 85: 8790-94 (1988) and FRET and TR-FRET have been described in for example U.S. Pat. Nos. 4,927,923; 5,220,012; 5,432,101; 5,457,185; 5,534,622; 5,346,996; 5,162,508; 5,512,493; 5,627,074; 5,527,684; 5,998,146; and, 6,291,201. Reagents useful for FRET, TR-FRET, HTRF are commercially available from vendors such as Cisbio International, Bedford, Mass.; Photon Technology International, Birmingham, N.J.; Invitrogen, La Jolla, Calif., GE Healthcare, Piscataway, N.J.

In any of the aforementioned aspects and embodiments of either the heterogeneous or homogeneous assay, the Keap1 protein or Keap1-kelch domain polypeptide can have an amino acid sequence from any species; however, it is generally preferred that the Keap1 protein or Keap1-kelch domain polypeptide have the amino acid sequence of the human KEAP1. The amino acid sequence for the human Keap1 protein is available from GenBank under accession number NP_{—}987096 and NP_{—}036421. In a currently preferred embodiment, the Keap1-kelch domain polypeptide comprises amino acid residues 322 to 609 of the human Keap1 protein. In particular embodiments, the Keap1 protein or Keap1-kelch domain polypeptide can be fused to a heterologous protein or polypeptide, for example, the glutathione-S-transferase (GST), maltose binding protein, thioredoxin, green fluoresecent protein, biotin carboxyl carrier protein, c-myc, FLAG, polyhistidine, or the like.

In currently preferred embodiments, the Nrf2 peptide comprises at least the 14 amino acids shown in SEQ ID NOs:1 and 2. The Nrf2 peptide comprising the amino acid sequence QLDEETGEFLPIQ (SEQ ID NO:2) has a binding affinity for the kelch domain of about 130+/−41 nM. In particular embodiments, the Nrf2 peptide can be replaced with a peptide comprising the amino acid sequence of Nrf1 or Pgam5, which is capable of binding the kelch domain of the Keap1 protein (Zhang et al., 2006, J. Biochem. 399:373-85; Lo et al., 2006, J. Biol. Chem. Epub ahead of print). The Nrf1 peptide can comprise the amino acid sequence LLVDGETGESFPAQ (SEQ ID NO:7) and the Pgam5 peptide can comprise the amino acid sequence RKRNVESGEEELAS (SEQ ID NO:8). The Nrf1 and Pgam5 peptides have a binding affinity for the kelch domain of about 397+/−133 nM and 626+/−197 nM, respectively.

The following examples are intended to promote a further understanding of the present invention.

### Example 1

The synthesis of Nrf2 Peptide 1 having amino acid sequence LQLDEETGEF(2-I)LPIQ-OH (SEQ ID NO: 1) was carried out by solid phase peptide methodology.

Nrf2 Peptide 1 was synthesized on an ABI 433A peptide synthesizer (ABI, Foster City, Calif., USA) using the manufacturer's 0.25 mmol Fastmoc double coupling protocol with HATU (O-(7-azabenzotriazole-1-yl)-1,1,3,3-tetramethyluronium hexafluorophosphate) as the coupling reagent in 1-methyl-2-pyrrolidinone. The synthesis started with H— Gln(Trt)-Cl-Trt resin (EMD Novabiochem, EMD Biosciences, San Diego, Calif.). The protected amino acids were Fmoc-Ile, Fmoc-Pro, Fmoc-Leu, Fmoc-Phe(2-I) (2-iodophenylalanine), Fmoc-Glu(OtBu), Fmoc-Gly, Fmoc-Thr(tBu), Fmoc-Asp(OtBu), and Fmoc-Gln(Trt). Following assembly, the peptide was cleaved from the resin with 95% TFA, 2.5% water and 2.5% triisopropylsilane for three hours at room temperature. Following filtration, the filtrate containing the product was evaporated to dryness under reduced pressure at room temperature. The residue was triturated with anhydrous diethyl ether, filtered, and dissolved in 50% acetonitrile/water. This peptide solution was then lyophilized. The crude peptide was purified by RP-HPLC on a DELTAPAK C18 100 Å column (Waters, Milford, Mass., USA) using a linear A/B gradient. Solution A was 0.1% NH₄HCO₃ in water, and solution B was acetonitrile. Pure fractions, as determined by analytical RP-HPLC, were pooled and lyophilized. The correct mass of the peptide was confirmed by electrospray mass spectrometry. Amino acid analysis was performed to confirm peptide composition and a peptide content value.

To introduce the tritium label, Nrf2 Peptide 1 was stirred with catalysts 10% Pd/C and 5% Pd/CaCO₃ and tritium gas in DMF on a Tritium manifold for one hour. The reaction mixture was filtered and co-evaporated with ethanol in order to remove any exchangeable tritium. The crude product was purified by using a semi-preparative HPLC column (Synergy 4u, Fusion RP 80, 250×10 mm column, water containing 0.1% TFA: acetonitrile 75:25, flow rate 4 mL/min, UV=254 nm) to yield [³H] Peptide (7.25 mCi, SA=17.6 Ci/mmol as determined by LC/MS, 25% yield).

### Example 2

The synthesis of Nrf2 Peptide 2 having amino acid sequence NH₂-LQLDEETGEFLPIQ-OH (SEQ ID NO:2) was prepared as described for Nrf2 peptide 1 with the exception that protected amino acid Fmoc-Phe(2-I) was replaced with protected amino acid Fmoc-Phe.

### Example 3

Cloning and purification of recombinant human Keap1-kelch domain by PCR amplification was as follows.

The DNA encoding the Keap1-kelch domain was PCR amplified using a DNA template encoding the full-length Keap1. DNAs encoding the full-length Keap1 protein and Keap1-kelch domain polypeptide were amplified by PCR using as the template a DNA clone encoding the full-length Keap1 protein, which had been synthesized by PCR using overlapping oligonucleotides based on the published sequence (See for example, Zhang and Hannink, 2003, Mol. Cell. Biol. 23: 8137-8151). The human Keap1 nucleotide sequence is also available at GenBank NM_{—}203500. The primers for amplifying DNA encoding the full-length Keap1 protein were 5′KFL-Nde1, 5′-GGGcatatgA TGCAGCCAGA TCCCAGG-3′ (SEQ ID NO:3) and 3′ KFL BamHI, 5′-CCGggatccT CAACAGGTAC AG-3′ (SEQ ID NO:4). The DNA encoding the Keap1-kelch domain was amplified using primers 5′KK-Nde1-2,5′-ATGCCCTGCC GcatatgGCG CCCAAGGTG-3′ (SEQ ID NO:5) and 3′ KK BamHI-new, 5′-CGggatccGG TGACAGCCAC GCCCAC-3′ (SEQ ID NO:6). The PCR conditions were as follows: initial denaturation at 94° C. for 10 minutes followed by 35 cycles of 94° C. for 30 seconds, 62° C. for 30 seconds, 72° C. for one minute with a final extension of five minutes at 72° C.

The amplified PCR DNA products were then cloned in pET15b vector (Catalog No. 70755-3, EMD Novabiochem, EMD Biosciences, San Diego, Calif.) between the NdeI and BamHI sites, to produce plasmid pET15b-Keap1-kelch domain. The cloned Keap1-kelch domain polypeptide was in frame with nucleotide codons encoding six histidines, which produced a recombinant Keap1-kelch domain polypeptide with a His tag at the N terminus of the Keap1-keltch domain. The authenticity of the nucleotide sequence encoding the recombinant Keap1-kelch domain polypeptide was confirmed by DNA sequencing. A cDNA encoding the full-length Keap1 proteins, which had been provided by Mark Hannink at the University of Missouri at Columbia (Zhang and Hannink, 2003, Mol. Cell. Biol. 23: 8137-8151), was used to provide full-length Keap1 protein for binding assays.

To overexpress the recombinant Keap1-kelch domain polypeptide, Rosetta2 BL21 PLysS cells (Cat. No. 200131, Stratagene, la Jolla, Calif.) were transformed with the pET15b-Keap1-kelch domain using plates that have 34 μg/mL chloramphenicol and 100 μg/mL ampicillin. All colonies were scraped into 10 to 20 milliliters of growth media. This stock culture was diluted into multiple flasks containing 1 liter each of growth media (NH₄Cl, 1 g; KH₂PO₄, 1 g; K₂HPO₄, 3 g; Na₂SO₄, 0.3 g; MgCl₂, 0.05 g; CaCl₂, 0.005 g, per liter) and 0.3% glucose supplemented with chloramphenicol (34 ug/mL) and Ampicillin (200 ug/mL)) so that the OD600 nanometers is at or below 0.05. The culture was grown at 37° C. with vigorous shaking until it reached an OD600 of 0.4 to 0.5. The cultures were cooled down to 25° C. by placing the flask on ice. The plasmid construct was induced for expression by IPTG (isopropyl β-D-1-thiogalactopyranoside) to a final concentration of 1 mM and gently shaking for 24 hours at 15° C., 1 mM PMSF (phenylmethylsulfonyl fluoride) was added simultaneously with the IPTG to reduce proteolysis of the expressed protein. The cultures were incubated at 25° C. for another five hours and cells were harvested by centrifugation at 5000×g for 15 minutes in a centrifuge.

The cell pellet was resuspended in 10 mL of lysis buffer (200 mM Tris-HCl pH 8.0, 500 mM sodium chloride, 10 mM Imidazole) containing fresh 1 mM PMSF and 2 mM β-mercaptoethanoland protease inhibitor cocktail mix without EDTA. The cell pellet was frozen at −80° C. and subsequently thawed at 4° C. twice.

The DNA present in the lysate was sheared by sonication on crushed ice and the supernatant fraction was separated from cell debris by centrifugation at 30,000×g. The supernatant fraction was FPLC purified by passing through a HITRAP Q HP 5 mL ion exchange column (GE Health Sciences, Inc., formerly Amersham, Piscataway, N.J.). The bound protein was eluted with an imidazole gradient to 200 mM Tris-HCl pH 8.0 buffer containing 500 mM sodium chloride and 500 mM imidazole. The major fractions containing recombinant Keap1-kelch domain were collected and dialyzed against 50 mM Tris-HCl, pH 8.0 containing 5 mM DTT. This protein was of sufficient purity to use in binding assays.

For crystallography, the major fractions containing recombinant Keap1-kelch domain polypeptide were collected and passed through a MONO Q anion exchange column (MONO Q is a trademark of GE Healthcare, Inc.) for further purification. The protein bound to the column was eluted with a 1M sodium chloride gradient (1M sodium chloride, 20 mM Tris-HCl pH 7.5, 5 mM DTT). The fractions corresponding to protein peaks containing recombinant Keap1-kelch domain polypeptide were collected and desalted by dialysis against 20 mM Tris-HCl pH 7.5 and 5 mM DTT. Subsequently, the pooled protein fractions were concentrated using Amicon concentrators and stored in aliquots at −80° C. in PBS containing 20% glycerol.

### Example 4

This example provides a protocol for an ion-based assay for identifying activators of the Nrf2-Keap1 system wherein a His-tagged Keap1 protein or Keap1-kelch domain polypeptide is immobilized to the surface of a 384-well plate via nickel divalent ions.

Stock solutions containing analytes at various concentrations or 500 nL dimethylsulfoxide (DMSO) and controls are transferred into the wells of a CHOICECOAT metal chelate white 384 well plates (Catalog No. NC15140 Pierce Biotechnology, Inc., Rockford, Ill.). The analyte volume is 500 nL per well. Controls include 100 μM unlabeled Peptide 2 and 0.5% DMSO.

An assay solution comprising 100 ng recombinant His-tagged Keap1 protein or Keap1-kelch domain polypeptide and 50 nM tritium labeled Nrf2 Peptide 2 in 50 μL of phosphate-buffered saline (PBS) per well is prepared. About 50 μL of the prepared solution is dispensed into each well of the 384 well plates. The plates are covered with foil seals and incubated at room temperature for two hours.

After the incubation, the plates are cooled by incubating in a 4° C. refrigerator for one hour. During the last 15 minutes of the 4° C. incubation, one L of cold PBS-T is added to the Liquid 1 wash bottle of the EMBLA 96/384 plate washer (Molecular Devices Corporation, Sunnyvale, Calif.). The instrument is primed using program ‘A1’ a minimum of three times.

Assay plates are removed from the refrigerator and washed using an EMBLA 96/384 plate washer. The EMBLA 96/384 system is programmed to aspirate the well, add 80 uL of PBS-T (phosphate buffered saline-0.1% Triton-X), aspirate the well, add 80 μL of PBS-T, and aspirate the well.

About 50 μL of MICROSCINT scintillation fluid (trademark of PerkinElmer Life and Analytical Sciences, Inc., Boston, Mass.) is dispensed into each of the wells of the assay plate and the assay plate is covered with a transparent adhesive seal. Counts per minute are recorded (Packard BioScience Company) and percent inhibition calculated for each compound relative to DMSO (no activity) and a 100 μM solution of Peptide-2 (100% inhibition).

Shown in FIGS. 1A and 1B are typical results, which were obtained using the above protocol using Keap1-kelch domain polypeptide and an analyte such as Analyte A (FIG. 1A) or the unlabeled Nrf2 Peptide 2 control (FIG. 1B). The unlabeled Peptide 2 control demonstrates the desired result expected for an analyte that competes with the labeled Peptide 2 for binding to the kelch domain.

The nickel-based assay is dependent on the Keap1 protein or Keap1-kelch domain polypeptide remaining bound to the nickel on the surface of the plate during the course of the assay. If an analyte competes with the Keap1 protein or Keap1-kelch domain polypeptide for binding to the nickel on the assay plate, then the Keap1-kelch domain polypeptide bound to the labeled Peptide 2 will dissociate from the assay plate giving a false positive signal. To distinguish between analytes that displace the labeled Peptide 2 from the kelch binding domain and that compete with the binding of the Keap1 protein or Keap1-kelch domain polypeptide to the nickel on the plate surface, a second assay in which there is no dependence on binding to nickel to immobilize the Keap1 protein or Keap1-kelch domain polypeptide (or Keap1 protein) to the surface of the assay plate was developed. In this assay, which is shown in Example 5, the results shown for Compound A in FIG. 1A was likely caused by Compound A competing with Keap1-kelch domain polypeptide for binding to the nickel on the surface of the plates.

### Example 5

This example provides a protocol for an antibody-based binding assay for identifying activators of the Nrf2-Keap1 system. Because Keap1 protein's or Keap1-kelch domain polypeptide's binding to the assay plate surface is not dependent on divalent metal cations such as nickel, the antibody-based assay is also useful for determining whether the competitive effect observed in an ion-based assay was a result of the analyte competing with binding of the labeled Peptide 2 with the Keap1-kelch domain or with the binding of the Keap1 protein or Keap1-kelch domain polypeptide with divalent cations on the plate. In general, the assay is performed as follows.

One μg/mL of mouse-derived anti-His tag antibody (Catalog No. 35370, Quiagen, Valencia, Calif.) is added to anti-mouse IgG-coated white plates (Catalog No. 15234, Pierce Biotechnology, Inc.) and incubated at room temperature for two hours. The plate is then washed with four chamber volumes of PBS to remove any excess antibody.

An assay solution containing 500 ng Keap1-kelch protein or Keap1-kelch domain polypeptide and 200 nM tritium-labeled Peptide 2 in 100 μL of PBS (per well) is prepared and 100 μL of this assay solution is dispensed into each well of the 384 well plates. Stock solutions containing analytes at various concentrations or 500 nL DMSO and controls are transferred into the wells. Controls include 100 μL unlabeled Nrf2 Peptide 2 and 0.5% DMSO. The plates are covered and incubated at room temperature for two hours.

After the incubation, the plates are cooled by incubating in a 4° C. refrigerator for one hour. During the last 15 minutes of the 4° C. incubation, one L of cold PBS-T is added to the Liquid 1 wash bottle of the EMBLA 96/384 plate washer (Molecular Devices Corporation, Sunnyvale, Calif.). The instrument is primed using program ‘A1’ a minimum of three times.

Assay plates are removed from the refrigerator and washed using program ‘06’ on the EMBLA 96/384. Program 06 is set to aspirate the well, add 80 uL of PBS-T, aspirate the well, add 80 μL of PBS-T, and aspirate the well.

About 50 μL of MICROSCINT scintillation fluid is dispensed into each of the wells of the assay plate and the assay plate covered with a transparent adhesive seal. Counts per minute are recorded and percent inhibition calculated for each compound relative to DMSO (no activity) and a 100 μM solution of Peptide-2 (100% inhibition).

The veracity of the antibody-based assay was tested with Analyte A, Analyte B (an analyte that binds nickel), and Analyte C over a concentration range of about 1 nm to about 100 μM and using the Keap1-kelch domain polypeptide. The results of the assay are shown in FIG. 2. As shown in FIG. 2, Analyte A, which gave a positive result in FIG. 1A, gave positive but inconsistent results in the antibody-based binding assay. This suggested that in the nickel-based assay, Analyte A might have been competing with the Keap1-kelch domain polypeptide for binding to the nickel on the surface of the plate and not with the kelch domain for binding to the labeled Nrf2 Peptide 2. FIG. 2 also shows Analytes B and C did not compete with the labeled Peptide 2 for binding to the kelch domain. Using unlabeled Nrf2 Peptide 2 to compete with the labeled Nrf2 Peptide 2 for binding to the kelch domain shows the result from this assay expected for an analyte that competes with binding of the labeled Peptide 2 for binding to the kelch domain.

### Example 6

This example shows a counterscreen to the nickel-based binding assay that is amenable to high-throughput screening. In this assay, His tagged Green Fluorescent Protein (GFP) retention on a divalent metal cation surface is monitored in the presence of analytes.

Stock solutions containing analytes at various concentrations or 500 nL DMSO and controls are transferred into the wells of a CHOICECOAT metal chelate white 384 well plates (Catalog No. NC15140, Pierce Biotechnology, Inc.). The volume is 500 nL per well. Controls include 100 μM unlabeled Peptide 2 and 0.5% DMSO.

An assay solution containing one μg His tagged GFP protein in PBS (per well) is prepared and 50 μL of this assay solution is dispensed into each well of the 384 well plates. The plates are covered and incubated at room temperature for two hours. Following the room temperature incubation, the plates are cooled by incubating in a 4° C. refrigerator for one hour. The plates are then washed by aspirating the well, adding 80 uL of PBS-T, aspirating the well, adding 80 uL of PBS-T, and aspirating the well.

Fluorescence emission at 535 nanometers when excited at 405 nanometers is recorded and percent inhibition of binding of the GFP to the nickel on the surface of the plate is calculated for each analyte relative to DMSO (no activity), a 100 μM solution of Peptide 2 (0% inhibition), and Analyte B (100% inhibition).

FIG. 3 shows the results of a typical assay using analytes A B, and C and Peptide 2. Analytes that displace the His-tagged GFP from the plate are undesirable and result in diminished fluorescent readout and increased percent inhibition. Peptide 2 demonstrates the desired result from this assay (0% inhibitions) and Analyte B demonstrates 100% inhibition. Analyte A gave a positive result in this assay, indicating that the effect seen in the nickel binding assay was a result of its displacing the His-tagged Keap1-kelch domain polypeptide from the plate (undesirable) and not from disruption of the interaction between the kelch domain and Peptide 2 (desirable). Because peptide 2 displays no competitive activity in this assay, but analyte B, which is known to chelate metal and displace the His-tagged GFP, achieves 100% inhibition of binding of the His-tagged GFP to the nickel on the surface of the assay plate, demonstrates that this assay is a useful final step of the triage process.

### Example 7

This prophetic example illustrates a method for identifying activators of the Nrf2-Keap1 system using a homogeneous time resolved fluorometry (HTRF) assay performed essentially according to the directions of the manufacturer, Cisbio International, Bedford, Mass.

Briefly, the Keap1 protein or Keap1-kelch domain polypeptide is labeled with Europium cryptate and Nrf2 Peptide 2 is labeled with XL665 according to the protocol provided by the manufacturer. Stock solutions containing analytes at various concentrations or 500 nL DMSO and controls are transferred into the wells of 384 well plates. The volume is 500 nL per well. Controls include 100 μM unlabeled Peptide 2 and 0.5% DMSO.

An assay solution comprising 100 ng Keap1 protein or Keap1-kelch domain polypeptide and 50 nM labeled Nrf2 Peptide 2 in 50 μL of PBS per well is prepared. About 50 μL of the prepared solution is dispensed into each well of the 384 well plates and the fluorescence from the acceptor fluorophore is measured at 655 nm over time. Analytes that disrupt binding of Peptide 2 to the Keap1 protein or Keap1-kelch domain polypeptide cause a decrease in fluorescence emission at 665 nm, which indicates that analyte may be useful in treatments and therapies for oxidative stress-related disorders.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for identifying an agent that activates the Keap1-Nrf2-ARE pathway, which comprises:
(a) providing a mixture including a Keap1 protein or Keap1-kelch domain polypeptide and an Nrf2 peptide that is capable of binding the kelch domain of the Keap1 protein
(b) adding an analyte to be evaluated for its ability to activate the Keap1-Nrf2-ARE pathway to the mixture; and
(c) determining the amount of Nrf2 peptide bound to the Keap1 protein or Keap1-kelch domain polypeptide, wherein a decrease in the amount of the Nrf2 peptide bound to the Keap1 protein or Keap1-kelch domain polypeptide compared to the amount of the Nrf2 peptide bound to the Keap1 protein or Keap1-kelch domain polypeptide in the absence of the analyte indicates that the analyte activates the Keap1-Nrf2-ARE pathway.

2. The method of claim 1 wherein the Nrf2 peptide is radiolabeled.

3. The method of claim 1 wherein the Nrf2 peptide is labeled with one member of a donor-acceptor fluorophore pair and the Keap1 protein or Keap1-kelch domain polypeptide is labeled with the other member of the fluorophore pair and fluorescence resonance energy transfer (FRET) or time-resolved FRET (TR-FRET) is measured to determine the amount of Nrf2 peptide bound to the Keap1 protein or kelch domain.

4. The method of claim 1 wherein the Nrf2 peptide comprises an amino acid sequence selected from the group consisting of SEQ ID NO:1, SEQ ID NO:7, and SEQ ID NO:2.

5. (canceled)

6. (canceled)

7. The method of claim 1 wherein the Keap1 protein or Keap1-kelch domain polypeptide is a fusion protein.

8. The method of claim 1 wherein the method is performed using a heterogeneous format wherein the Keap1 protein or Keap1-kelch domain polypeptide is immobilized to the surface of a solid support.

9. The method of claim 6 wherein the Keap1 protein or kelch domain has a polyhistidine tag.

10. The method of claim 9 wherein the Keap1 protein or Keap1-kelch domain polypeptide having the polyhistidine tag is immobilized to the surface of the solid support via divalent metal ions.

11. The method of claim 9 wherein the Keap1 protein or Keap1-kelch domain polypeptide having the polyhistidine tag is immobilized to the surface of the solid support using antibodies specific for the polyhistidine tag which have been immobilized to the surface of the solid support.

12. The method of claim 1 wherein the method is performed using a homogeneous format wherein the Keap1 protein or Keap1-kelch domain polypeptide is labeled with one member of a donor-acceptor fluorophore pair and the Nrf2 peptide labeled with the other member of the fluorophore pair and fluorescence resonance energy transfer (FRET) or time-resolved FRET (TR-FRET) is measured to determine the amount of Nrf2 peptide bound to the Keap1 protein or kelch domain.

13. A method for identifying an analyte that is an activator of the Keap1-Nrf2-ARE pathway, which comprises:
(a) providing a first assay wherein a mixture of a Keap1 protein or Keap1-kelch domain polypeptide having a polyhistidine tag bound to a labeled Nrf2 peptide is immobilized to the surface of a first solid support via divalent metal ions and a second assay wherein a detectable protein having a polyhistidine tag is immobilized to the surface of a second solid support via divalent metal ions wherein the protein;
(b) adding an analyte to be evaluated for ability to activate the Keap1-Nrf2-ARE pathway to the first assay and the second assay; and
(c) determining the amount of the Nrf2 peptide bound to the Keap1 protein or Keap1-kelch domain polypeptide in the first assay in the presence of the analyte and the amount detectable protein immobilized to the second solid support in the presence of the analyte, wherein a decrease in the amount of the Nrf2 peptide bound to the Keap1 protein or Keap1-kelch domain polypeptide compared to the amount bound to the Keap1 protein or Keap1-kelch domain polypeptide in the absence of the analyte and no detectable change in the amount of detectable protein immobilized to the surface of the second support indicates that the analyte is an activator of the Keap1-Nrf2-ARE pathway.

14. The method of claim 13 wherein a third assay is provided in which the Keap1 protein or Keap1-kelch domain polypeptide having the polyhistidine tag bound to the labeled Nrf2 peptide is immobilized to the surface of a third solid support using antibodies specific for the polyhistidine tag wherein the antibodies have been immobilized to the surface of the third solid support;
(a) adding the analyte to be evaluated for ability to activate the Keap1-Nrf2-ARE pathway to the third assay; and
(c) determining the amount of the Nrf2 peptide bound to the Keap1 protein or Keap1-kelch domain polypeptide in the third assay in the presence of the analyte, wherein a decrease in the amount of the Nrf2 peptide bound to the Keap1 protein or Keap1-kelch domain polypeptide compared to the amount bound to the Keap1 protein or Keap1-kelch domain polypeptide in the absence of the analyte in the first and third assays and no detectable change in the amount of detectable protein immobilized to the surface of the second support indicates that the analyte is an activator of the Keap1-Nrf2-ARE pathway.

15. The method of claim 14 wherein the detectable protein is labeled with a fluorophore or has a detectable enzymatic activity.

16. (canceled)

17. (canceled)

18. A method for identifying an agent that activates the Keap1-Nrf2-ARE pathway, which comprises:
(a) providing mixture that includes a Keap1 protein or Keap1-kelch domain polypeptide labeled with one member of a donor-acceptor fluorophore pair and a Nrf2 peptide that is capable of binding the Keap1 protein or Keap1-kelch domain polypeptide labeled with the other member of the donor-acceptor pair, wherein the acceptor fluorophore produces a detectable fluorescence when the Keap1 protein or Keap1-kelch domain polypeptide is bound to the Nrf2 peptide;
(b) adding an analyte to be evaluated for its ability to activate the Keap1-Nrf2-ARE pathway to the mixture; and
(c) measuring the amount of the detectable fluorescence from the acceptor fluorophore over time wherein a decrease in the amount of detectable fluorescence from the acceptor fluorophore over time in the presence of the analyte indicates that the analyte activates the Keap1-Nrf2-ARE pathway.

19. The method of claim 17 wherein the donor fluorophore includes a lanthanide and time-resolved FRET (TR-FRET) is measured to determine the amount of Nrf2 peptide bound to the Keap1 protein or kelch domain.

20. The method of claim 16 wherein the lanthanide is Eu3+.

21. The method of claim 16 wherein the donor fluorophore is Europium cryptate.

22. The method of claim 16 wherein the Nrf2 peptide comprises an amino acid sequence selected from the group consisting of SEQ ID NO:1, SEQ ID NO:7, and SEQ ID NO:2.

23. (canceled)

24. (canceled)

25. The method of claim 16 wherein the Keap1 protein or Keap1-kelch domain polypeptide is a fusion protein.


SEQUENCE LISTING (8 sequences)

SEQ ID NO: 1 - protein, 14 aa, Artificial Sequence
  Nrf2 peptide
LQLDEETGEXLPIQ

SEQ ID NO: 2 - protein, 13 aa, Artificial Sequence
  Nrf2 peptide
QLDEETGEFLPIQ

SEQ ID NO: 3 - DNA, 27 bp, Artificial Sequence
  5′KFL-Nde1 PCR primer
gggcatatgatgcagccagatcccagg

SEQ ID NO: 4 - DNA, 22 bp, Artificial Sequence
  3′KFL-BamHI PCR primer
ccgggatcctcaacaggtacag

SEQ ID NO: 5 - DNA, 29 bp, Artificial Sequence
  5′KK-NdeI-2 PCR primer
atgccctgccgcatatggcgcccaaggtg

SEQ ID NO: 6 - DNA, 26 bp, Artificial Sequence
  3′KK-BamHI-new PCR primer
cgggatccggtgacagccacgcccac

SEQ ID NO: 7 - protein, 14 aa, Artificial Sequence
  Nrf1 peptide
LLVDGETGESFPAQ

SEQ ID NO: 8 - protein, 14 aa, Artificial Sequence
  Pgam5 peptide
RKRNVESGEEELAS